(19) United States
(12) Patent Application Publication
(10) Pub. No.: US 2024/0269429 A1
(43) Pub. Date: Aug. 15, 2024

(54) FLEXIBLE RADIOPAQUE CATHETER SHAFT

(71) Applicant: Medtronic, Inc., Minneapolis, MN (US)

(72) Inventors: Chandrasekhar Ramasubramanian Rajagopalan, Minneapolis, MN (US); Juan Pablo Garcia, Aguas Buenas, PR (US); Nathan Edward Cozine, St. Paul, MN (US)

(21) Appl. No.: 18/168,275

(22) Filed: Feb. 13, 2023

(51) Int. Cl. A61M 25/00

(52) U.S. Cl. CPC A61M 25/0053; A61M 25/0012; A61M 25/0045; A61M 2025/0007

(57) ABSTRACT

A flexible, radiopaque catheter shaft is provided. The catheter shaft includes a first tubing, a second tubing, and a reinforcement layer disposed between the first tubing and the second tubing. The reinforcement layer may include, for example, radiopaque multifilament formed in a braid or a coil.

### BACKGROUND

The present disclosure is generally directed to shafts, and relates more particularly to flexible, radiopaque catheter shafts that are able to resist or prevent sharp bends (e.g., kinks or buckles).

Catheters can be used for various functions. In some cases, catheters are used to sample fluid from a patient. In other cases, catheters may be used to deliver fluids, therapeutics or medical devices to the patient. Catheter shafts of conventional catheters may be heavy, may be radiolucent, may form a sharp bend (e.g., a kink, or buckle) when bent, and/or may not be flexible.

### BRIEF SUMMARY

Example aspects of the present disclosure include:

A fluid system according to at least one embodiment of the present disclosure comprises a port implanted in a patient; and a catheter implanted in the patient and in fluid communication with the port, the catheter comprising a catheter shaft having a first tubing, a second tubing, and a reinforcement layer disposed between the first tubing and the second tubing.

Any of the aspects herein, wherein the catheter is implanted to sample fluid from at least one of a spinal region or a brain region of the patient.

Any of the aspects herein, further comprising a pump configured to at least one of pump a fluid sample to the port via the catheter or pump a therapeutic to the patient from the port via the catheter.

Any of the aspects herein, wherein the pump is implanted in the patient.

Any of the aspects herein, wherein the reinforcement layer comprises multifilament yarn formed in at least one of a braid or a coil.

Any of the aspects herein, wherein the multifilament yarn may comprise at least one of polyethylene, polypropylene, polyesters, or polyamides.

Any of the aspects herein, wherein the multifilament comprises radiopaque filler, the radiopaque filler comprising at least one of barium, tungsten, tantalum, or bismuth.

Any of the aspects herein, wherein the multifilament is formed in the braid and wherein the braid comprises at least one of a one-over-one pattern, two-over-two pattern, or three-over-three pattern.

Any of the aspects herein, wherein the reinforcement layer comprises a first layer and a second layer, the first layer and the second layer formed from different materials.

Any of the aspects herein, wherein each of the first tubing and the second tubing comprises at least one of silicone, polyurethane, polyamide, polyester, fluoropolymers, or copolymers.

A fluid system according to at least one embodiment of the present disclosure comprises a port implanted in a patient; and a catheter implanted in the patient and in fluid communication with the port, the catheter comprising a catheter shaft having a first tubing, a second tubing, and a reinforcement layer disposed between the first tubing and the second tubing, the reinforcement layer comprising radiopaque multifilament formed in at least one of a braid or a coil.

Any of the aspects herein, wherein the radiopaque multifilament is formed in a braid, wherein the braid comprises at least one of a one-over-one pattern, two-over-two pattern, or three-over-three pattern.

Any of the aspects herein, wherein the reinforcement layer comprises a first layer and a second layer, the first layer and the second layer formed from different materials.

Any of the aspects herein, further comprising a pump configured to at least one of pump a fluid sample to the port via the catheter or pump a therapeutic to the patient from the port via the catheter.

Any of the aspects herein, wherein the catheter is implanted to sample fluid from at least one of a spinal region or a brain region of the patient.

Any of the aspects herein, wherein each of the first tubing and the second tubing comprise silicone.

A catheter shaft according to at least one embodiment of the present disclosure comprises a first tubing comprising silicone; a second tubing comprising silicone; and a reinforcement layer disposed between the first tubing and the second tubing, the reinforcement layer comprising radiopaque multifilament formed in a braid; wherein the first tubing forms an outer layer and the second tubing forms an inner layer.

Any of the aspects herein, wherein the reinforcement layer comprises a first layer and a second layer, the first layer and the second layer formed from different materials.

Any of the aspects herein, wherein the braid comprises at least one of a one-over-one pattern, two-over-two pattern, or three-over-three pattern.

Any of the aspects herein, wherein the catheter is implanted to sample fluid from at least one of a spinal region or a brain region of the patient.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a block diagram of a system according to at least one embodiment of the present disclosure;

FIG. 3 is a block diagram of a fluid system according to at least one embodiment of the present disclosure;

FIG. 4 is a block diagram of a fluid system according to at least one embodiment of the present disclosure;

FIG. 5 is an isometric view of a catheter shaft according to at least one embodiment of the present disclosure;

FIG. 6 is a front view of the catheter shaft of FIG. 5;

FIG. 7 is an isometric view of a catheter shaft according to at least one embodiment of the present disclosure;

FIG. 8 is a front view of the catheter shaft of FIG. 7;

FIG. 9 is an X-ray image of a catheter shaft according to at least one embodiment of the present disclosure; and

FIG. 10 is a side view of a bent catheter shaft according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Conventional catheter shafts may be formed from reinforced composite shaft designs to obtain shafts of multilayer tubing for use with, for example, catheters and/or lead bodies. In some embodiments, the catheter may be used in systems for sampling fluids and/or delivering fluids or therapeutics to a patient. However, such designs may lack flexibility, kink-resistance, and/or toughness. For example, filled polymer tubing may increase toughness of a catheter, but also increase its stiffness and decrease long-term stability. In another example, metal wire braids may increase toughness and kink resistance, but interact with magnetic fields and result in shafts with low flexibility. In another example, metal wires as coils may be difficult to manufacture for thin-walled, multilayer shafts and may be limited in inner diameter sizes. Further, conventional designs for thin-walled shafts may not be radiopaque or may not appear clearly in X-ray images without having high content (by weight) of the radiopaque ingredient, which could negatively impact performance characteristics such as stiffness and long-term stability.

According to at least one embodiment of the present disclosure, a catheter having a shaft reinforced by radiopaque multifilament yarns may be used to form reinforced braids disposed between an outer tubing and an inner tubing. Such braids formed from radiopaque multifilament yarns may enable a catheter shaft that is flexible, tough, kink-resistant, and radiopaque. Further, the first tubing and the second tubing may prevent the braids from being exposed to the environment (e.g., a patient anatomy), thereby maintaining a durability of the braid and/or maintaining or increasing a biological safety of the catheter.

Embodiments of the present disclosure provide technical solutions to one or more of the problems of (1) increasing a visibility of a catheter in imaging and particularly, in X-ray imaging (2) increase toughness and durability of a catheter, and (3) increase a catheter's resistance to forming sharp bends (e.g., kinks or buckles).

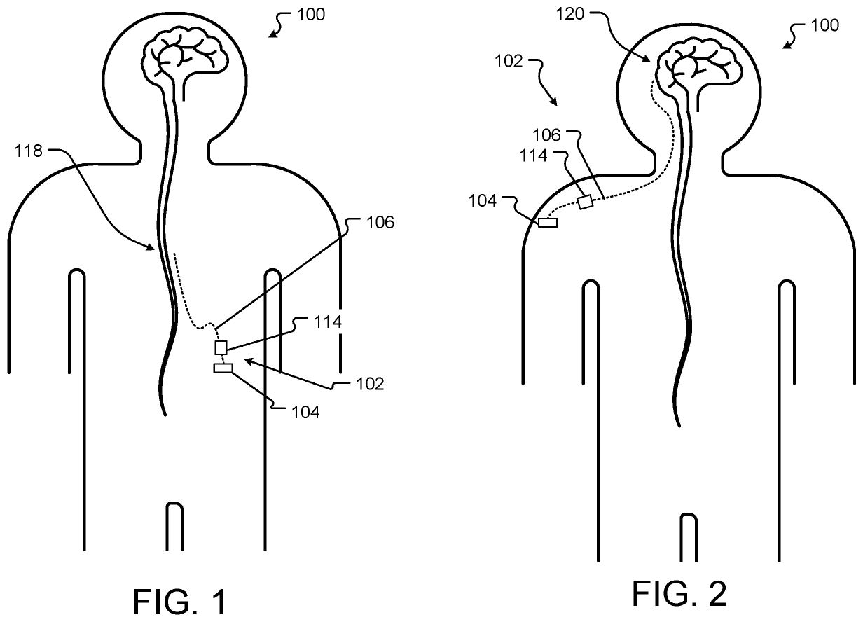

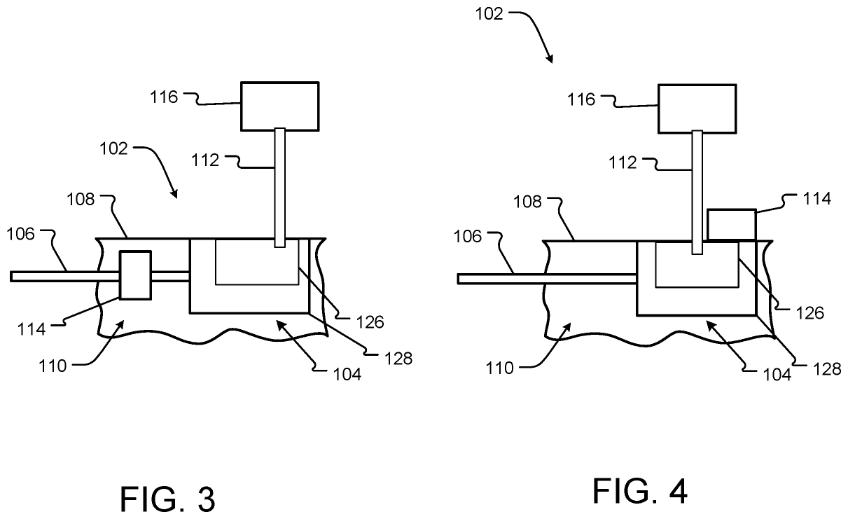

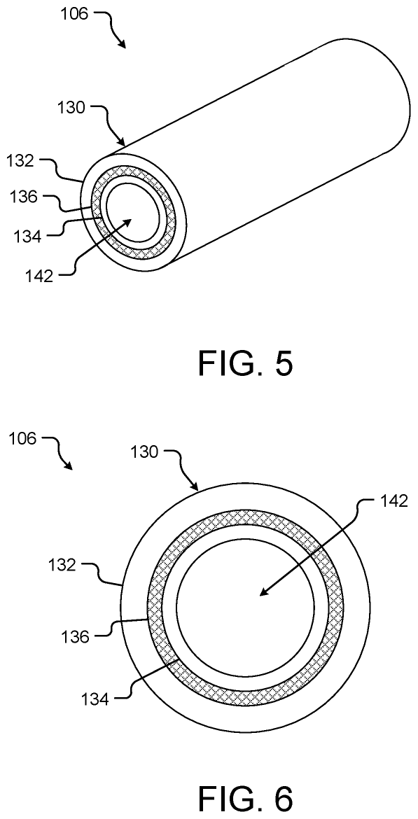

Turning first to FIG. 1, a block diagram of a patient 100 and a fluid system 102 according to at least one embodiment of the present disclosure is illustrated. It will be appreciated that the fluid system 102 enables fluid sampling from the patient 100 and/or delivery of therapeutics to the patient 100. In some embodiments, the fluid system 102 can collect fluid sample from, for example, an arterial space or vascular space. The fluid system 102 may comprise a catheter 106 (also shown and discussed in detail in FIGS. 5-10), a pump 114, and a port 104 having a septum 126. It will be appreciated that in some embodiments, the fluid system 102 may not include the pump 114. The catheter 106 may enable fluid sampling from the patient 100 and/or delivery of the fluid sample via the port 104 and the pump 114 may be configured to pump the fluid sample to the port 104 via the catheter 106. The catheter 106 may also enable delivery of therapeutics to the patient 100 and the pump 114 may be configured to pump the therapeutics to the patient 100 via the port 104 and the catheter 106. The fluid system 102 may include additional components such as, for example, a needle 112 (shown in FIGS. 3 and 4), a syringe, a reservoir 116 (shown in FIGS. 3 and 4), or any other component.

Some or all components of the fluid system 102 may be implanted in the patient 100. In the illustrated embodiment, the catheter 106, the pump 114, and the port 104 are implanted in the patient 100, though in other embodiments in which the fluid system 102 includes the pump 114, the pump 114 may not be implanted in the patient 100 (as described in FIG. 4). In the illustrated embodiment, the catheter 106 may be implanted in a spinal region 118 of the patient 100. In other embodiments, the catheter 106 may be implanted anywhere in the patient 100. In the illustrated embodiment the pump 114 and the port 104 are implanted near an abdomen of the patient 100, though it will be appreciated that the pump 114 and the port 104 may be implanted anywhere in the patient 100.

Turning to FIG. 2, a block diagram of the patient 100 and the fluid system 102 according to at least one embodiment of the present disclosure is illustrated. FIG. 2 is substantially similar to FIG. 1 except for placement of the fluid system 102. In the illustrated embodiment, the catheter 106 is implanted near a brain region 120 of the patient 100 to enable sampling of, for example, cerebral spinal fluid. In such embodiments, the pump 114 and/or the port 104 may be positioned along an arm region of the patient 100. It will be appreciated that in other embodiments, the catheter 106, the pump 114, and/or the port 104 may be implanted or positioned anywhere on the patient 100.

Turning to FIGS. 3 and 4, a block diagram of the fluid system 102 in a first configuration and a second configuration are shown respectively. As described above, the fluid system 102 comprises the catheter 106, the pump 114, and the port 104. The catheter 106 may be any catheter 106 that is implantable in a patient 100 and enables fluid sampling from the patient 100 and/or delivery of therapeutics to the patient 100. As previously described, the catheter 106 may be implanted anywhere in a patient 100 including, for example, intrathecally in the spinal region 118 of the patient 100, in the brain region 120 of the patient 100, etc. Though the catheter 106 is described as a component of the fluid system 102, it will be appreciated that a catheter shaft 130 (shown in FIGS. 5-10) of the catheter 106 may be used in other applications such as, for example, cardiovascular delivery catheters, balloon catheters and/or as a jacket or shaft for stimulation leads. Thus, the catheter shaft 130 can be used in any application in which a flexible, tough, kink-resistant shaft or jacket is desired and/or beneficial.

The fluid system 102 may also include the pump 114. In some embodiments, the pump 114 may be or comprise the Medtronic SynchroMed™ Pump. In other embodiments, the pump 114 may be or comprise any pump 114 configured to pump sampling fluid from the patient 100 or therapeutics to the patient 100. As previously described, the pump 114 may be implanted in the patient 100, as shown in FIG. 3, or positioned outside of the patient 100, as shown in FIG. 4. In some embodiments, the fluid system 102 may not include the pump 114.

The fluid system 102 also includes the port 104. The port 104 comprises a housing 128 and a septum 126 disposed in the housing 128. The septum 126 may be in fluid communication with the catheter 106. In the illustrated embodiment, the port 104 is implanted subcutaneously 110 under a surface 108 of the patient's 100 skin. In other embodiments, the port 104 may be implanted anywhere on the patient 100 and may be implanted at the surface 108 or above the surface 108 of the patient's 100 skin. The port 104 is in fluid communication with the catheter 106 and provides for easy and repeatable access to fluid samples from the patient 100. Similarly, the port 104 provides for easy and repeatable access to the patient 100 to deliver therapeutics. The port 104 may be in fluid communication with the catheter 106 via one or more connectors. In some embodiments, the port 104 can be coupled to the catheter 106 via the one or more connectors and without sutures. The port 104 may also comprise a port as described in patent application Ser. Nos. 17/085,682; 17/085,562; 17/556,571; and/or 16/949,418, which are incorporated by reference in their entireties.

In some embodiments, the fluid system 102 may also include a needle 112 configured to access the port 104. In embodiments where the port 104 is implanted below the surface 108 of the patient's 100 skin, the needle 112 is configured to pierce the patient's skin and the septum 126 until the needle 112 contacts a needle stop in the port beneath the septum 126. Once the needle 112 has reached the stop, the needle 112 is in fluid communication with the port 104 to collect the fluid sample or to deliver therapeutics. In other embodiments, where the port 104 may be accessible at or above the surface 108 of the patient's 100 skin, the needle 112, a syringe, or other component may be used to access the port 104 via the septum 126. The needle 112 (or syringe) may couple to a reservoir 116 which may collect the fluid sample from the port 104 or deliver therapeutics to the port 104. In other embodiments, the reservoir 116 may be integrated or positioned near the pump 114. For example, the pump 114 may automatically obtain fluid samples periodically and store such fluid samples in the reservoir 116. In other examples, the pump 114 may automatically deliver therapeutics to the patient from the reservoir 116. It will be appreciated that in some embodiments that the fluid sampling system 102 may not include the reservoir 116. For example, in some embodiments, a syringe may be coupled to the port 104 (whether directly or via the needle 112) and may collect the fluid sample or deliver the therapeutics.

Turning to FIGS. 5 and 6, an isometric view and a front view of at least one embodiment of the catheter 106 are respectively shown. The catheter 106 may comprise the catheter shaft 130 formed from a first tubing 132, a reinforcement layer 136, and a second tubing 134. The catheter shaft 130 also includes a bore 142 through which fluids and/or therapeutics are delivered therethrough. In the illustrated embodiment, the first tubing 132 forms an outer layer of the catheter shaft 130, the second tubing 134 forms an inner layer of the catheter shaft 130, and the reinforcement layer 136 is disposed between the first tubing 132 and the second tubing 134. In some embodiments, the first tubing 132 and the second tubing 134 may be formed from silicone. In other embodiments, the first tubing 132 and the second tubing 134 may be formed from any type of material such as, for example, thermoplastics, elastomers, thermoplastic elastomers, polyurethane, polyamide, polyester, fluoropolymers, or copolymers incorporating silicone, polyurethane, polyamide, polyester, and/or fluoropolymers, etc. It will be appreciated that the first tubing 132 and the second tubing 134 may be formed from the same material or may be formed from different materials. The first tubing 132 and/or the second tubing 134 may include radiopaque filler to achieve improved radiopacity. The radiopaque filler may include, for example, barium, tungsten, tantalum, bismuth, or the like. The radiopaque filler may be used in a metallic form, an oxide form, a sulfate form, or any other inorganic form. In some embodiments, the first tubing 132, the second tubing 134, and the reinforcement layer 136 may include radiopaque filler or materials. The first tubing 132 and the second tubing 134 provide a sterile, smooth surface such that the catheter 106 can be implanted in the patient 100 and fluids and/or therapeutics can be retrieved or delivered therethrough.

The reinforcement layer 136 provides reinforcement to the catheter shaft 130 and enables the catheter shaft 130 to be flexible, tough, and able to resist sharp bends (e.g., kinks or buckles). More specifically, the reinforcement layer 136 provides toughness when stretched and enables a softer profile when flexing to prevent sharp bends (e.g., kinks or buckles) when the reinforcement layer 136 is bent. In some embodiments the reinforcement layer 136 is formed from a multifilament sold under the trademark DYNEEMA®. It will be appreciated that the reinforcement layer 136 may be formed from any multifilament such as, for example, polyethylene, polypropylene, polyesters such as polyethylene terephthalate, polyamides, etc. The multifilament may also include radiopaque filler, which may enable the catheter 106 to be, for example, tracked or detected by X-ray imaging.

The radiopaque filler may comprise, for example, barium, tungsten, tantalum, bismuth, or the like and the radiopaque filler may be used in a metallic form, an oxide form, a sulfate form, or any other inorganic form. In some instances, the multifilament yarn may comprise two or more monofilaments in a braid pattern. The braid pattern of the two or more monofilaments may be any braid pattern such as, for example, a one-over-one pattern, a two-over-two pattern, a three-over-three pattern, etc.

In some embodiments, the multifilament (which may have any number of monofilaments in any braid pattern) may be braided in a one-over-one pattern over, for example, the second tubing 134. In such embodiments, the multifilament may be braided in any pattern such as, for example, a two-over-two pattern, a three-over-three pattern, etc. In other embodiments, the multifilament may be coiled. In still other embodiments, the reinforcement layer 136 may comprise multiple materials such as, for example, a combination of multifilament, polymers, metals, or any combination thereof. The multiple materials may be formed into braids and/or coils.

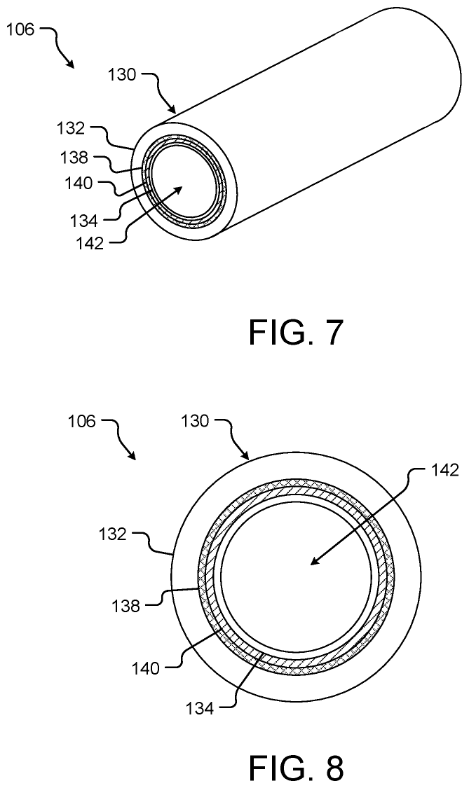

Turning to FIGS. 7 and 8, an isometric view and a front view of another embodiment of the catheter 106 according to the present disclosure are respectively shown. The catheter 106 as shown comprises a catheter shaft 130 that is substantially similar to the catheter shaft 130 shown in FIGS. 5 and 6. However, the reinforcement layer 136 comprises a first layer 138 and a second layer 140. It will be appreciated that in other embodiments the reinforcement layer 136 may comprise any number of layers. The first layer 138 and the second layer 140 may be formed from the same material. In other instances, the first layer 138 and the second layer 140 may be formed from different materials. Similarly, the first layer 138 and the second layer 140 may be formed in the same pattern (e.g., a braid in a one-over-one pattern, a braid in a two-over-two pattern, a braid in a three-over-three pattern, a coil, etc.) and in other instances, the first layer 132 and the second layer 140 may be formed in different patterns. The multiple layers may provide additional reinforcement to the catheter 106.

It will be appreciated that in some instances, the catheter shaft 130 may comprise any number of layers of reinforcement layers and/or tubing. In some embodiments, the catheter shaft 130 may have more than two tubing and more than two reinforcement layers. For example, the catheter shaft 130 may comprise a first, outer tubing, a first reinforcement layer, a second, midway tubing, a second reinforcement layer, and a third, inner tubing. In another example, the reinforcement layer may have more than two layers.

Further, in some embodiments, the catheter shaft 130 may be manufactured using heat shrink. In other embodiments, the catheter shaft 130 may be manufactured by expanding with pressure and/or temperature and/or solvent, for example, the first tubing 132 and positioning the first tubing 132 over the reinforcement layer 136. It will be appreciated that the catheter shaft 130 can be formed or manufactured in a number of ways and using any combination of manufacturing processes.

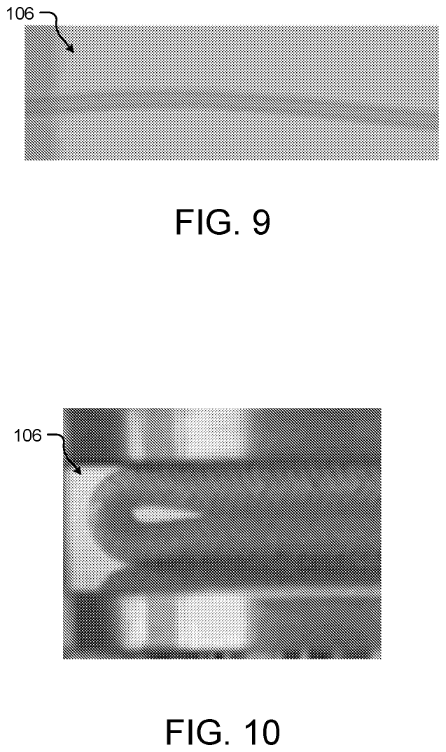

Turning to FIGS. 9 and 10, an example of the radiopaque and flexible properties of the catheter shaft 130 are shown respectively. As shown in FIG. 9, in embodiments where the reinforcement layer 136 includes radiopaque materials such as, for example, radiopaque multifilament, the reinforcement layer 136 is visible and detectable in X-ray imaging. As previously described, this may enable the tracking and/or monitoring of the catheter 106 during, for example, insertion or implantation of the catheter 106, placement of the catheter 106, and/or removal of the catheter 106. As shown in FIG. 10, the catheter 106 is shown in a bent configuration in which the catheter 106 is able to bend 180 degrees without forming a sharp bend, whereas a conventional catheter may typically form a sharp bend when bent to such degrees. Thus, the catheter 106 as described in the present disclosure beneficially provides a catheter that is durable and can withstand implantation within a patient, that is flexible to avoid or prevent sharp bends from forming in the catheter, and is radiopaque and thus can be tracked or monitored via X-ray imaging.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A fluid system comprising:
a port implanted in a patient; and
a catheter implanted in the patient and in fluid communication with the port, the catheter comprising a catheter shaft having a first tubing, a second tubing, and a reinforcement layer disposed between the first tubing and the second tubing.

2. The system of claim 1, wherein the catheter is implanted to sample fluid from at least one of a spinal region or a brain region of the patient.

3. The system of claim 1, further comprising a pump configured to at least one of pump a fluid sample to the port via the catheter or pump a therapeutic to the patient from the port via the catheter.

4. The system of claim 3, wherein the pump is implanted in the patient.

5. The system of claim 1, wherein the reinforcement layer comprises multifilament yarn formed in at least one of a braid or a coil.

6. The system of claim 5, wherein the multifilament yarn may comprise at least one of polyethylene, polypropylene, polyesters, or polyamides.

7. The system of claim 5, wherein the multifilament comprises radiopaque filler, the radiopaque filler comprising at least one of barium, tungsten, tantalum, or bismuth.

8. The system of claim 5, wherein the multifilament is formed in the braid and wherein the braid comprises at least one of a one-over-one pattern, two-over-two pattern, or three-over-three pattern.

9. The system of claim 1, wherein the reinforcement layer comprises a first layer and a second layer, the first layer and the second layer formed from different materials.

10. The system of claim 1, wherein each of the first tubing and the second tubing comprises at least one of silicone, polyurethane, polyamide, polyester, fluoropolymers, or copolymers.

11. A fluid system comprising:
a port implanted in a patient; and
a catheter implanted in the patient and in fluid communication with the port, the catheter comprising a catheter shaft having a first tubing, a second tubing, and a reinforcement layer disposed between the first tubing and the second tubing, the reinforcement layer comprising radiopaque multifilament formed in at least one of a braid or a coil.

12. The system of claim 11, wherein the radiopaque multifilament is formed in a braid, wherein the braid comprises at least one of a one-over-one pattern, two-over-two pattern, or three- over-three pattern.

13. The system of claim 11, wherein the reinforcement layer comprises a first layer and a second layer, the first layer and the second layer formed from different materials.

14. The system of claim 11, further comprising a pump configured to at least one of pump a fluid sample to the port via the catheter or pump a therapeutic to the patient from the port via the catheter.

15. The system of claim 11, wherein the catheter is implanted to sample fluid from at least one of a spinal region or a brain region of the patient.

16. The system of claim 11, wherein each of the first tubing and the second tubing comprise silicone.

17. A catheter shaft comprising:
a first tubing comprising silicone;
a second tubing comprising silicone; and
a reinforcement layer disposed between the first tubing and the second tubing, the reinforcement layer comprising radiopaque multifilament formed in a braid;
wherein the first tubing forms an outer layer and the second tubing forms an inner layer.

18. The catheter shaft of claim 17, wherein the reinforcement layer comprises a first layer and a second layer, the first layer and the second layer formed from different materials.

19. The catheter shaft of claim 17, wherein the braid comprises at least one of a one-over-one pattern, two-over-two pattern, or three-over-three pattern.

20. The catheter shaft of claim 17, wherein the catheter is implanted to sample fluid from at least one of a spinal region or a brain region of the patient.
